(19) United States
(12) Patent Application Publication
(10) Pub. No.: US 2017/0290750 A1
(43) Pub. Date: Oct. 12, 2017

(54) PIGMENT MIXTURE

(71) Applicant: MERCK PATENT GMBH, Darmstadt (DE)

(72) Inventors: Lukas HAMM, Otzberg (DE); Nicole HAFERKORN, Pfungstadt (DE); Marita JEKEL, Darmstadt (DE)

(73) Assignee: MERCK PATENT GMBH, Darmstadt (DE)

(21) Appl. No.: 15/484,387

(22) Filed: Apr. 11, 2017

(30) Foreign Application Priority Data
Apr. 11, 2016 (DE) 102016004164.1

(51) Int. Cl. A61K 8/25; A61K 8/26; A61Q 1/10; A61Q 1/12; A61Q 1/02; A61Q 19/00; A61Q 3/02; A61Q 1/06; C23C 16/44; C23C 16/40; C23C 16/00; C23C 14/22; C23C 14/08; C23C 14/00; B01J 2/00; A61K 8/02

(52) U.S. Cl. CPC A61K 8/25; A61K 8/025; A61K 8/26; A61Q 1/10; A61Q 1/12; A61Q 1/02; A61Q 19/007; A61Q 3/02; A61Q 1/06; C23C 16/4417; C23C 16/40; C23C 16/006; C23C 14/223; C23C 14/08; C23C 14/0015; B01J 2/006; A61K 2800/43

(57) ABSTRACT

The present invention relates to a pigment mixture based on at least two components A and B, where
component A is a mixture of flake-form and spherical substrates which is covered with one or more inorganic layers and/or organic layers,
   and
component B comprises crystalline or amorphous particles selected from the group of the metal oxides, metal hydroxides, metal oxyhalides, Prussian Blue or mixtures thereof,
   and to the use thereof in paints, coatings, printing inks, security printing inks, plastics, ceramic materials, glasses, in cosmetic formulations, as tracer, as filler and for the preparation of pigment preparations and dry preparations.

The present invention relates to a pigment mixture based on at least three components, and to the use thereof in paints, coatings, printing inks, security printing inks, plastics, ceramic materials, glasses, in cosmetic formulations, as tracer, as filler and for the preparation of pigment preparations and dry preparations.

Matt absorptive pigments are generally employed in cosmetic formulations in order to obtain a mass tone of the formulation. These have to be pre-dispersed in a complex manner in order to incorporate them into cosmetic formulations. If matt absorptive pigments are employed, these often have a dull and chalky effect in cosmetic formulations, meaning that the skin takes on a dry appearance.

Fillers can be regarded as a special form of pigments. In the case of fillers, the “coloring” function is not at the forefront. In the case of industrial fillers, factors such as the improvement of skin feel, the increase in the mechanical stability, the abrasion resistance, the weather stability or also the production costs are instead crucial for their use.

The fillers based on SiO₂ spheres which are known from the prior art exhibit a relatively good skin feel, but have the disadvantage that they have an excessively high scattering capacity. The reason for this can be found in the structure of the metal-oxide layers of the spherical fillers. The functional pigments from the prior art generally consist of very small particles, i.e. they have particle sizes of 0.5-100 nm, which cover the surface of the carrier spheres in a uniform arrangement. Light is reflected at the layer surface, causing gloss. At the same time, however, a considerable degree of scattering occurs since individual particles form on the layer and act as strong centers of scattering. As a consequence of these two opposing effects (gloss and scattering), the pigments have a white and unnatural appearance on the skin.

In addition, the conventional fillers are generally colorless or weakly colored and not very opaque. Thus, if fillers are added to a system which also comprises absorptive pigments and/or effect pigments, the fillers both influence the effect and also reduce the tinting strength and the hiding power.

Fillers are also widely used in cosmetic formulations. For example, powders may comprise up to 50% of fillers, based on the final formulation. Typical values are 10-15% of fillers in lipsticks and 2-6% of fillers in emulsions. Cosmetic fillers fulfil completely different functions: in foundations, they prevent an undesired greasy sheen on the skin due to the so-called matting effect, while in powders they help, for example, to improve the pouring behavior or the application properties to the skin. In deodorant products, the high liquid absorption capacity of some fillers is utilized.

Before their use in the system to be pigmented, fillers or pigments in cosmetics have to be brought into a form which facilitates easy dispersion and a reproducible color. These pretreatments of the pigments, for example grinding, which have a crucial influence on the quality of the end product are time-consuming and expensive. It is furthermore disadvantageous that the color of the pigment is changed on wetting. For cosmetic formulations, the pigments must additionally have a good skin feel, which the classical absorptive pigments only exhibit to a small extent.

Fillers based on spherical particles, in particular SiO₂ spheres, are increasingly being employed in cosmetics, since they impart a natural appearance on human skin on the one hand and can make wrinkles less visible on the other hand.

Inorganic spherical fillers which are covered with a coloring layer are known, for example, from the published specifications JP 62-288662, JP 11-139926, JP 11-335240 and DE 199 29 109.

WO 00/15720 and EP 2826822 A1 disclose pigment mixtures based on spherical SiO₂ particles and/or flake-form substrates.

WO 99/66883 describes SiO₂ spheres which are coated with metal oxides, such as titanium oxide, iron oxide or zinc oxide, and have a final SiO₂ layer. The SiO₂ spheres coated in this way are employed in cosmetic formulations as a mixture with interference pigments.

Absorptive pigments, such as, for example, iron oxide pigments, frequently exhibit poor dispersibility in cosmetic formulations, a dry and dull appearance of the skin and/or result in an unsatisfactory skin feel. In order to overcome these disadvantages, various fillers are frequently employed or the iron oxide pigments are subjected to a pretreatment in order to enable them to be dispersed in the cosmetic formulations, which is associated with expenditure of time and costs.

An object of the present invention is therefore to provide a functional and homogeneous pigment mixture which, besides a good skin feel, simultaneously has good dispersibility in cosmetic formulations, chemical and photochemical stability and a pure color matched to the skin as well as a high hiding power and does not have the above-mentioned disadvantages.

In addition, the pigment mixture, when applied to the skin as a pure powder, in creams, emulsions, foundations and the like, should exhibit a soft and uniform and natural appearance of the skin. It is furthermore desired for the pigment to impart a slight increase in the firmness, in particular of liquid and pasty preparations, and to guarantee the stability of the preparation. This makes it significantly easier to distribute the cosmetic preparations on the skin. Thus, high-viscosity creams, i.e. solid foundations, can be prepared which nevertheless have very good distribution ability on the skin or very good removal behavior on removal from the container.

Besides these product properties, a further object of the invention is simple industrial preparation of the pigment mixture. It should also be possible to monitor the setting of certain properties, such as, for example, the hiding power and the specifically adjustable color intensity, of the pigment mixture in a simple manner in the course of the preparation process.

Surprisingly, a pigment mixture in the form of a three-component system has been found which does not have the above-mentioned disadvantages, but instead is distinguished by a very good skin feel and high hiding power and imparts a fresh and natural appearance on the skin, since matt texture and slight luster or brightness are specifically combined with one another.

The present invention relates to a pigment mixture comprising components A and B, where
component A is a mixture of flake-form and spherical substrates which is covered with one or more inorganic layers and/or organic layers, and
component B comprises crystalline or amorphous particles selected from the group of the metal oxides, metal hydroxides, metal oxyhalides, Prussian Blue (Berlin Blue) or mixtures thereof.

The three-component mixture according to the invention is distinguished over the pigments and pigment mixtures from the prior art by
a matt appearance with defined luster and/or defined color intensity
a more natural color-matched appearance of the skin
easier dispersibility
improved processability
a hiding power which can be adjusted within broad limits
an improved skin feel
an improved texture of the cosmetic preparations
an improved application behavior of the cosmetic formulations
a broadened color space which is evident in the case of matt formulations
a homogeneous powder, since there is no separation into the individual components.

A further advantage of the pigment mixture according to the invention over the products from the prior art is the homogeneity of the powder. Whereas separation often occurs in the case of separate use of fillers, absorptive pigments and effect pigments, a homogeneous product is ensured by the process according to the invention, i.e. all components of the pigment mixture according to the invention are inseparably bound to one another and separation into the individual components is not possible.

Furthermore, the pigment mixture according to the invention exhibits the desired influence on the texture and stability, i.e. it slightly increases the viscosity of emulsions or the firmness of foundations without adversely affecting the application properties, and at the same time it maintains the stability of the preparation.

The invention furthermore relates to the use of the pigment mixture according to the invention in paints, coatings, preferably in industrial coatings, printing inks, security printing inks, plastics, ceramic materials, glasses, as tracer, as filler and in particular in cosmetic formulations. Furthermore, the pigments according to the invention are also suitable for the preparation of pigment preparations and for the preparation of dry preparations, such as, for example, granules, pearlets, chips, pellets, sausages, briquettes, etc. The dry preparations are used, in particular, in printing inks and in the cosmetics field.

The flake-form substrates of component A are preferably transparent flake-form substrates. Preferred substrates are phyllosilicates. Particularly suitable are natural or synthetic mica, talc, kaolin, flake-form Fe₂O₃, Fe₃O₄, Al₂O₃, BiOCl, glass, SiO₂, TiO₂, BN, oxynitride, nitride and graphite flakes, pearl essence, synthetic support-free flakes or other comparable materials.

It is also possible to employ mixtures of different flakes.

Particularly preferred flake mixtures of component A comprise or consist of
mica flake+SiO₂ flake
mica flake+Al₂O₃ flake
mica flake+glass flake
mica flake+TiO₂ flake
mica flake+oxynitride flake
mica flake+nitride flake
mica flake+pearl essence
mica flake+graphite flake
mica flake+BiOCl
SiO₂ flake+Al₂O₃ flake
glass flake+SiO₂ flake.

The size of the flake-form substrates is not crucial per se and can be matched to the respective application. In general, the flake-form substrates have a thickness between 0.05 and 1.5 μm, in particular between 0.1 and 1 μm. The size in the other two dimensions is usually between 1 and 250 μm, preferably between 2 and 200 μm, and in particular between 5 and 60 μm. It is also possible to employ substrates of different particle sizes. Particular preference is given to a mixture of mica fractions of N mica (10-60 μm), F mica (5-20 μm) and M mica (<15 μm). Preference is furthermore given to N and S fractions (10-130 μm) and F and S fractions (5-130 μm).

In a very particularly preferred embodiment, the flake-form substrates of component A have a particle size of 0.1-100 μm, in particular 0.3-60 μm and very particularly preferably 0.5-15 μm.

Suitable spherical base substrates for component A are spherical particles, as are, for example, commercially available, inter alia from Sunjin Chemicals, Kobo, Ikeda, Asahi Glass, Miyoshi, Omega Materials, 3M, ABC NanoTech, China New Technology, PQ Corporation, Sibelco or Evonik. Preferred spherical particles are selected from the group magnesium silicate, aluminum silicate, alkali-metal aluminum silicates, alkaline-earth metal aluminum silicates and combinations thereof, SiO₂ spheres, glass beads, hollow glass beads, aluminum oxide, ceramic beads and polymeric beads comprising ethylene-acrylic acid copolymers, ethylene-methacrylate copolymers, HDI-trimethylol hexyl lactone copolymers, nylon, polyacrylates, polymethyl methacrylate copolymers, polyethylene, polymethylsilsesquioxanes and combinations thereof.

Particularly preferred spherical substrates are SiO₂ spheres, as are commercially available, for example, under the trade names SUNSIL, MSS-500, SHERON, SUNSPHERE, Silicabeads SB, OMEGA-SIL, OMEGA-Spheres, SPHERICEL, Q-Cel, Ceramic Microspheres, Glasbubbles iM-K, SILNOS, SS-T4, SS-S3, SORBOSIL, AEROSIL, Flo-Beads, BPD, Daiamid, MSP, TOSPEARL and Ronaspheres.

The BET surface area, determined by nitrogen absorption, of the suitable spherical base particles is generally 1-1000, preferably 10-750, in particular 20-550 m²/g. The BET surface area in this patent application is determined in accordance with DIN ISO 9277: 2003-05.

The spherical base substrates of component A preferably have a particle diameter in the range 0.1-100 μm, in particular 0.3-60 μm and very particularly preferably 0.5-15 μm.

The spherical and flake-form substrates of component A can be mixed with one another in any mixing ratio. The spheres:flakes ratio is preferably 99:1 to 1:99, in particular 90:10 to 10:90, very particularly preferably 85:15 to 25:75. The luster and color intensity can be increased by an increased proportion of flakes, while a greater proportion of spheres has an advantageous effect on the skin feel and the homogeneity when applied.

Particularly preferred substrate mixtures of component A are given below:
natural mica flakes+SiO₂ spheres
synthetic mica flakes+SiO₂ spheres
Al₂O₃ flakes+SiO₂ spheres
SiO₂ flakes+SiO₂ spheres
glass flakes+SiO₂ spheres
natural mica flakes+glass spheres
synthetic mica flakes+glass spheres
Al₂O₃ flakes+glass spheres
SiO₂ flakes+glass spheres
glass flakes+glass spheres.

The mixture of spherical and flake-form particles of component A is subsequently covered with one or more, preferably one or two layers, preferably inorganic layers. The inorganic layer(s) preferably comprise absorbent and non-absorbent oxides or Prussian Blue.

The inorganic layer preferably comprises or consists of oxides selected from the group Fe₂O₃, Fe₃O₄, FeOOH, Cr₂O₃, ZrO₂, Al₂O₃, SnO₂, SiO₂, ZnO, TiO₂ and titanium suboxides. The TiO₂ here can be in the rutile or anatase modification.

The inorganic layers are preferably absorbent layers of Fe₂O₃, Fe₃O₄, Cr₂O₃, FeOOH, TiO₂/Fe₂O₃ mixed layer, pseudobrookite, ilmenite, Prussian Blue or Carmine Red.

In a preferred embodiment, component A has at least one absorbent layer and at least one non-absorbent layer on the surface of the flake/sphere mixture.

The thickness of the individual layers on the substrate mixture of component A is essential for the optical properties of the final product. In particular, the 1st layer on the surface of the sphere/flake mixture has an essential influence on the color properties.

The thickness of the first layer is preferably 10-1000 nm, in particular 30-600 nm and particularly preferably 50-300 nm. The layer thicknesses of the 1st, 2nd and further layers, if present, may be identical or different. They preferably have similar or identical layer thicknesses and may also be exchanged in the sequence.

With the aid of the coating (one or more layers), it is possible to vary or adjust the color, luster and hiding powder in broad limits.

Particularly preferred mixtures (spheres+flakes) of component A have the following layer sequences on the surface:
substrate mixture+Fe₂O₃ (1st layer)+TiO₂ (2nd layer)
substrate mixture+Fe₂O₃ (1st layer)+SnO₂ (2nd layer)
substrate mixture+Fe₂O₃ (1st layer)+SiO₂ (2nd layer)
substrate mixture+Fe₃O₄ (1st layer)+TiO₂ (2nd layer)
substrate mixture+Fe₃O₄ (1st layer)+SnO₂ (2nd layer)
substrate mixture+Fe₃O₄ (1st layer)+SiO₂ (2nd layer)
substrate mixture+Cr₂O₃ (1st layer)+TiO₂ (2nd layer)
substrate mixture+Cr₂O₃ (1st layer)+SnO₂ (2nd layer)
substrate mixture+Cr₂O₃ (1st layer)+SiO₂ (2nd layer)
substrate mixture+TiO₂ (1st layer)+Prussian Blue (2nd layer)
substrate mixture+TiO₂ (1st layer)+Carmine Red (2nd layer)
substrate mixture+SnO₂ (1st layer)+TiO₂ (2nd layer)
substrate mixture+TiO₂ (1st layer)
substrate mixture+Fe₂O₃ (1st layer)
substrate mixture+FeOOH (1st layer)
substrate mixture+TiO₂ (1st layer)+Fe₂O₃ (2nd layer)
substrate mixture+Prussian Blue (1st layer)
substrate mixture+Cr₂O₃ (1st layer)
substrate mixture+Fe₂O₃ (1st layer)+TiO₂ (2nd layer)+SiO₂ (3rd layer)
substrate mixture+TiO₂ (1st layer)+SiO₂ (2nd layer)+TiO₂ (3rd layer)

Component B preferably comprises crystalline or amorphous particles from the group Carmine Red, Prussian Blue, metal oxide, such as, for example, TiO₂, Fe₂O₃, Cr₂O₃, Fe₃O₄, titanium suboxides, such as, for example, TiOₓ, where x=1.5-1.95, metal hydroxide, such as, for example, FeOOH, metal oxyhalide, such as, for example, BiOCl, or mixtures thereof.

Component B preferably has particle sizes in the range 0.1-100 μm, in particular 0.3-30 μm and very particularly preferably 0.5-10 μm.

Particular preference is given to pigment mixtures in which all base substrates, i.e. flakes, spheres of component A, and the crystalline or amorphous particles of component B, have similar particle sizes. Especial preference is given to pigment mixtures where the three base substrates of components A and B all have a particle size or a particle diameter in the range 0.1-100 μm, in particular 0.3-30 μm and very particularly preferably 0.5-15 μm.

The pigment mixture according to the invention can easily be prepared by covering the substrate mixture of component A with one or more layers, for example metal oxide layer(s), Prussian Blue and mixing component A with component B. The covering of the substrate mixture of component A is preferably carried out by wet-chemical methods. The aqueous suspension of component B is then added to the aqueous suspension of component A. However, it is also possible to separate off and work up the product after covering of the substrate mixture of component A and subsequently to mix the dried product of component A with the suspension of component B. A further possibility consists in preparing the suspension of a component B, subsequently adding the substrate mixture of component A and then covering this mixture in a one-pot synthesis.

The metal-oxide layers on the surface of the substrate mixture of component A are preferably applied by wet-chemical methods, where the wet-chemical coating methods developed for the preparation of pearlescent pigments can be used. Methods of this type are described, for example, in U.S. Pat. No. 3,087,828, U.S. Pat. No. 3,087,829, U.S. Pat. No. 3,553,001, DE 14 67 468, DE 19 59 988, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017, DE 196 18 568, EP 0 659 843, or also in further patent documents and other publications known to the person skilled in the art.

In the case of wet coating, the spherical and flake-form base substrates of component A are suspended in water, and one or more hydrolysable metal salts are added at a pH suitable for hydrolysis, which is selected so that the metal oxides or metal oxide hydrates are precipitated directly onto the flakes and spheres without secondary precipitations occurring. The pH is usually kept constant by simultaneous metered addition of a base or acid. Component B, preferably as a suspension in water, is subsequently added to the substrate mixture coated in this way (=component A) and mixed, and the final product is separated off, washed and dried and optionally calcined, where the calcination temperature can be optimized with respect to the coating present in each case. In general, the calcination temperatures are between 250 and 900° C., preferably between 450 and 700° C. If desired, the substrate mixture of component A can be separated off, dried or calcined after application of individual coatings and then resuspended again for precipitation of the further layers.

If titanium dioxide and iron oxide are employed in the pigment mixture according to the invention, hues in yellow, brown and red can be achieved, depending on the calcination temperature. In particular at calcination temperatures>700° C., the yellow shades predominate, since in the case of component A, mixed oxides comprising titanium oxide and iron oxide, for example pseudobrookite, form.

The Fe₃O₄ layer can be produced, for example, by reduction of the Fe₂O₃ layer using ammonia, hydrogen or also hydrocarbons and hydrocarbon/ammonia mixtures, as described, for example, in EP-A-0 332 071, DE 19 51 696.8 and DE 19 51 697.7. The reduction is preferably carried out in a forming gas atmosphere (N₂/H₂), in particular at 92% of N₂/8% of H₂ or 96% of N₂/4% of H₂. The reduction temperature is preferably 400 to 700° C., in particular 500 to 600° C.

For the application of a final SiO₂ layer, the process described in DE 196 18 569 is preferably used. For the production of the SiO₂ layer, sodium or potassium water-glass solution is preferably employed.

Furthermore, the coating can also be carried out by gas-phase coating in a fluidized-bed reactor, where, for example, the processes proposed in EP 0 045 851 and EP 0 106 235 for the preparation of pearlescent pigments can be used correspondingly.

The hue of the final pigment mixture can be varied within broad limits through the different choice of the coverage rates or the layer thicknesses resulting therefrom. Fine tuning for a certain hue can be achieved, beyond the pure choice of amounts, by approaching the desired color under visual or measurement-technology control.

Joint work-up of the suspension comprising component A and component B enables the properties indicated above to be established and at the same time ensures homogeneity of the pigment mixture and prevents separation due to the particle shape and the structured surface of the coated substrates of the pigment mixture.

The invention also relates to the processes for the preparation of the pigment mixture according to the invention.

The invention furthermore relates to pigment mixtures prepared by the processes according to the invention, which are distinguished by the fact that, in the case of wet-chemical coating, the aqueous suspension of component B is added to the aqueous suspension of component A and the suspension comprising components A and B is worked up, or in that, after wet-chemical covering of the substrate mixture of component A, the product is separated off and worked up and the dried product of component A is subsequently mixed with the suspension of component B and the suspension is then worked up, or in that the substrate mixture of component A is added to the suspension of component B, and the mixture of component B and the substrate mixture of component A is jointly covered with one or more organic or inorganic layers by wet-chemical methods and then worked up.

Coating(s) in this patent application is taken to mean the complete covering of the respective surface of the base substrates.

Component A and component B can be mixed with one another in any ratio. For color optimization in the respective application, the preferred mixing ratio (parts by weight) of component A to component B is 99:1 to 1:99, in particular 90:10 to 10:90 and very particularly preferably 80:20 to 20:80. The mixing ratio here relates to the weight.

The pigment mixture according to the invention generally has an oil absorption value of 10-200 g /100 g, in particular 20-200 g /100 g, very particularly preferably 50-150 g /100 g. The oil absorption value in this patent application is determined in accordance with DIN ISO 787/5-1980 (E).

The pigment mixtures according to the invention improve, in particular, the texture of cosmetics by achieving easier application and more uniform distribution on the skin and improving the skin feel. Since the pigment mixture according to the invention is built up on a non-toxic mineral basis and comprises predominantly inorganic components, it is very well tolerated on the skin.

In order to increase the light, water and weather stability, it is frequently advisable, depending on the area of application, to subject the pigment mixture to post-coating or post-treatment. Suitable post-coating or post-treatment methods are, for example, those described in German patent 22 15 191, DE-A 31 51 354, DE-A 32 35 017 or DE-A 33 34 598.

This post-coating further increases the chemical and photochemical stability or makes handling of the pigment mixture, in particular incorporation into various media, easier. In order to improve the wettability, dispersibility and/or compatibility with the application media, functional coatings comprising A1₂O₃ or ZrO₂ or mixtures thereof can be applied to the pigment surface. Furthermore, organic post-coatings are possible, for example with silanes, as described, for example, in EP 0090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff. and P. H. Harding, J. C. Berg, J. Adhesion Sci. Technol. Vol. 11 No. 4, pp. 471-493.

The invention also relates to a process for the preparation of the pigment mixture according to the invention in which flake-form substrates are mixed with spherical particles and preferably covered with one or more absorbent or non-absorbent layers, for example metal oxides, by wet-chemical methods, and the coated substrate/sphere mixture (component A) is mixed with crystalline or amorphous particles (component B), and the mixture of components A and B is worked up jointly, and the final mixture is dried and optionally calcined at temperatures of 150-1,000° C.

The pigment mixture according to the invention is compatible with a multiplicity of color systems, preferably from the area of paints, coatings and printing inks. A multiplicity of binders, in particular water-soluble products, as marketed, for example, by BASF, Marabu, ProII, Sericol, Hartmann, Gebr. Schmidt, Sicpa, Aarberg, Siegwerk, GSB-Wahl, Follmann, Ruco or Coates Screen INKS GmbH, are suitable for the preparation of printing inks for, for example, gravure printing, flexographic printing, offset printing or offset overprint varnishing. The printing inks can be water-based or solvent-based.

Particularly effective effects, such as smoothing of surfaces and levelling-out of unevenness (wrinkles, pores, recesses, microcracks, etc.), can be achieved with the pigment mixture according to the invention in the various application media, for example in cosmetic formulations, such as, for example, nail varnishes, lipsticks, compact powders, gels, lotions, soaps, toothpaste, in paints, in industrial coatings and powder coatings, and in plastics and in ceramics.

Owing to the good skin feel and the very good skin adhesion, the pigment mixture according to the invention is particularly suitable as filler in decorative cosmetics, but also for personal care applications, such as, for example, body lotions, emulsions, soaps, shampoos, BB creams, CC creams, DD creams, etc. The pigment mixture according to the invention has a stabilizing action, as is desired, for example, in creams, emulsions and lotions.

On use in plastics, for example in injection-molded parts, the use has a positive effect on the prevention of flow lines or sink marks.

It goes without saying that the pigment mixture according to the invention can also advantageously be employed for the various applications as a blend with, for example,
metal-effect pigments, for example based on iron flakes or aluminum flakes;
pearlescent pigments based on metal oxide-coated synthetic mica flakes, natural mica flakes, glass flakes, Al₂O₃ flakes, Fe₂O₃ flakes or SiO₂ flakes;
interference pigments based on metal oxide-coated synthetic mica flakes, natural mica flakes, glass flakes, Al₂O₃ flakes, Fe₂O₃ flakes or SiO₂ flakes;
goniochromatic pigments;
multilayered pigments (preferably comprising 2, 3, 4, 5 or 7 layers) based on metal oxide-coated synthetic mica flakes, natural mica flakes, glass flakes, Al₂O₃ flakes, Fe₂O₃ flakes or SiO₂ flakes;
organic dyes;
organic pigments;
inorganic pigments, such as, for example, transparent and opaque white, colored and black pigments;
flake-form iron oxides;
carbon black.

The pigment mixture according to the invention can be mixed in any ratio with commercially available pigments and/or further commercially available fillers.

Commercially available fillers which may be mentioned are, for example, natural and synthetic mica, nylon powder, pure or filled melamine resins, talc, glasses, kaolin, oxides or hydroxides of aluminum, magnesium, calcium, zinc, BiOCl, barium sulfate, calcium sulfate, calcium carbonate, magnesium carbonate, carbon, boron nitride and physical or chemical combinations of these substances. There are no restrictions with respect to the particle shape of the filler. It can be, for example, flake-shaped, spherical or needle-shaped, in accordance with requirements.

The pigment mixture according to the invention can of course also be combined in the formulations with any type of cosmetic raw materials and assistants. These include, inter alia, oils, fats, waxes, film formers, preservatives and assistants which generally determine applicational properties, such as, for example, thickeners and rheological additives, such as, for example, bentonites, hectorites, silicon dioxides, Ca silicates, gelatins, high-molecular-weight carbohydrates and/or surface-active assistants, etc.

The formulation comprising the pigment mixture according to the invention can belong to the lipophilic, hydrophilic or hydrophobic type. In the case of heterogeneous formulations having discrete aqueous and non-aqueous phases, the pigment mixture according to the invention may be present in only one of the two phases in each case or alternatively distributed over both phases.

The pH values of the formulations can be between 1 and 14, preferably between 2 and 11 and particularly preferably between 4 and 10.

No limits are set for the concentrations of the pigment mixture according to the invention in the formulation. They can be—depending on the application—between 0.001 (rinse-off products, for example shower gels) and 60%. The pigment mixture according to the invention may furthermore also be combined with cosmetic active compounds. Suitable active compounds are, for example, insect repellents, inorganic UV filters, such as, for example, TiO2, UV A/BC protection filters (for example OMC, B3, MBC), anti-ageing active compounds, vitamins and derivatives thereof (for example vitamin A, C, E, etc.), self-tanning agents (for example DHA, erythrulose, inter alia) and further cosmetic active compounds, such as, for example, bisabolol, LPO, ectoin, emblica, allantoin, bioflavonoids and derivatives thereof.

Organic UV filters are generally employed in an amount of 0.5-10% by weight, preferably 1-8% by weight, inorganic UV filters in an amount of 0.1-30% by weight, based on the cosmetic formulation.

In addition, the formulations may comprise further conventional skin-protecting or skin-care active ingredients, such as, for example, aloe vera, avocado oil, coenzyme Q10, green tea extract and also active-compound complexes.

The present invention likewise relates to formulations, in particular cosmetic formulations, which, besides the pigment mixture according to the invention, comprise at least one constituent selected from the group of absorbents, astringents, antimicrobial substances, antioxidants, antiperspirants, antifoaming agents, antidandruff active compounds, antistatics, binders, biological additives, bleaches, chelating agents, deodorizers, emollients, emulsifiers, emulsion stabilizers, dyes, humectants, film formers, fillers, fragrances, flavors, insect repellents, preservatives, anticorrosion agents, cosmetic oils, solvents, oxidants, vegetable constituents, buffer substances, reducing agents, surfactants, propellant gases, opacifiers, UV filters and UV absorbers, denaturing agents, aloe vera, avocado oil, coenzyme Q10, green tea extract, viscosity regulators, perfume and vitamins.

The invention also relates to the use of the pigment mixture according to the invention in paints, coatings, printing inks, security printing inks, plastics, ceramic materials, glazes, glasses, as tracer, in cosmetic formulations and for the preparation of pigment preparations and dry preparations.

The following examples are intended to explain the invention in greater detail, but without restricting it.

In the foregoing and in the examples, all temperatures are set forth uncorrected in degrees Celsius and, all parts and percentages are by weight, unless otherwise indicated.

### EXAMPLES

### Example 1

### Pigment Mixture Having a Blue Mass Tone

### Batch 1A:

8 g of natural mica flakes from Merck KGaA (particle size<15 μm) together with 80 g of silicon dioxide spheres from ABC Nanotech (diameter: 2-4 μm) are stirred into 800 ml of deionized water.

This suspension is heated to the reaction temperature of 75° C. with stirring. The pH is adjusted to 2.1 by means of 10% sulfuric acid, and 2200 g of a 25% titanium tetrachloride solution are then metered in. The pH is kept constant using 20% sodium hydroxide solution. When the addition is complete, the mixture is stirred for a further 5 minutes, and the pH is then adjusted to 4.3 using 10% sulfuric acid. Two solutions 1 and 2 are subsequently added simultaneously, while the pH is kept constant using a 10% ammonium carbonate solution. Solution 1 is prepared from 60 g of potassium hexacyanoferrate (III) and 1300 g of deionized water. Solution 2 consists of 76 g of iron sulfate heptahydrate and 1250 g of deionized water. When the addition is complete, the mixture is stirred for a further 15 min, and the pH is then set to pH 6 using 20% sodium hydroxide solution.

### Batch 1B:

900 ml of deionized water are heated to the reaction temperature of 73° C. with stirring, and the pH is then adjusted to 4.3 using 10% sulfuric acid. Two solutions 1 and 2 are subsequently added simultaneously, while the pH is kept constant using a 10% ammonium carbonate solution. Solution 1 is prepared from 165 g of potassium hexacyanoferrate (III) and 2100 g of deionized water. Solution 2 consists of 209 g of iron sulfate heptahydrate and 1950 g of deionized water. The pH is then set to pH 6 using 20% sodium hydroxide solution.

When both batches are complete, the suspensions of batches 1A and 1B are mixed, filtered off, washed and dried at 150° C. and then sieved through a sieve having a mesh width of 24 μm, giving a pigment mixture having a blue mass tone and a soft skin feel and matt texture which has a high chroma and can be incorporated extremely well into cosmetic formulations without further effort. The hiding power, skin feel and chroma can be adjusted as desired through the 1A/1B mixing ratio.

### Example 1 a

If the 1A :1B ratio is 8:2, particularly highly chromatic pigment mixtures having an extraordinarily good skin feel and a moderate hiding power are obtained.

### Example 1 b

If the 1A:1B ratio is 1:1, particularly highly chromatic pigment mixtures having a very good skin feel and medium hiding power are obtained.

### Example 1 c

If the 1A:1B ratio is 2:8, highly chromatic pigment mixtures having a very high hiding power and a good skin feel are obtained.

### Example 2

### Pigment Mixture Having a Red Mass Tone

### Batch 2A:

8 g of synthetic mica flakes from Merck KGaA (particle size<15 μm) together with 80 g of silicon dioxide spheres from Sinoenergy (diameter 2-4 μm) are stirred into 800 ml of deionized water.

This suspension is heated to the reaction temperature of 73° C. with stirring, and the pH is then adjusted to 3.3 using 10% hydrochloric acid. 923 g of 10% iron chloride solution are subsequently metered in, during which the pH is kept constant using 20% sodium hydroxide solution. When the addition is complete, the mixture is stirred for a further 5 minutes. The pH is then set to pH 2.1 using 10% HCl, and 310 g of 25% titanium tetrachloride solution are subsequently metered in. During this, the pH is kept constant using 20% sodium hydroxide solution. When the addition is complete, the mixture is stirred for a further 5 minutes. The pH is then set to pH 7 using 20% sodium hydroxide solution, and the mixture is stirred for a further 15 min.

### Batch 2B:

1.7 l of deionized water are heated to the reaction temperature of 73° C. with stirring, and the pH is then adjusted to 3.3 using 10% hydrochloric acid. 1000 g of 10% iron chloride solution are subsequently metered in, during which the pH is kept constant using 20% hydroxide solution. When the addition is complete, the mixture is stirred for a further 5 minutes, and the pH is then set to pH 7 using 20% sodium hydroxide solution.

When both batches are complete, the suspensions of batches 2A and 2B are mixed, filtered off, washed and dried at 150° C. The pigment mixture is subsequently calcined at 780° C. and then passed through a sieve having a mesh width of 24 μm.

A pigment mixture having a burgundy mass tone and a soft skin feel and matt texture is obtained which has a high chroma and can be incorporated extremely well into cosmetic formulations without further effort. The hiding power, skin feel and chroma can be adjusted as desired through the 2A/2B mixing ratio.

### Example 2a

If the 2A :2B ratio is 8:2, particularly highly chromatic pigment mixtures having an extraordinarily good skin feel and a moderate hiding power are obtained.

### Example 2b

If the 2A :2B ratio is 1:1, particularly highly chromatic pigment mixtures having a very good skin feel and a medium hiding power are obtained.

Example 2c: If the 2A :2B ratio is 2:8, highly chromatic pigment mixtures having a very good hiding power and a good skin feel are obtained.

### Example 3

### Pigment Mixture Having a White Mass Tone

1.2 l of deionized water are heated to the reaction temperature of 75° C. with stirring, and the pH is then adjusted to pH 1.8 using 10% hydrochloric acid. 20 ml of a titanium tetrachloride solution (400 g/l) are subsequently metered in over the course of 25 minutes, during which the pH is kept constant using 20% sodium hydroxide solution. When the addition is complete, the mixture is stirred for a further 20 minutes, and 600 ml of a suspension of 20 g of natural mica flakes from Merck KGaA (particle size<15 μm) and 60 g of silicon dioxide spheres from ABC Nanotech (diameter 2-4 μm) in water are metered in over the course of 30 minutes, during which the pH is kept constant using hydrochloric acid. 200 ml of a titanium tetrachloride solution (400 g/l) are subsequently metered in over the course of 2.5 hours, during which the pH is kept at 1.8 using sodium hydroxide solution. After a subsequent stirring time, the pH is set to 5 using sodium hydroxide solution, and the mixture is stirred for a further 15 minutes.

The batch is filtered off, and the filter residue containing the product is washed with water and dried at 150° C. The powder is subsequently calcined at 800° C. for 45 minutes and then sieved through a sieve having a mesh width of 24 μm.

A pigment mixture having a white mass tone, a soft skin feel, a matt texture and an elegant shimmer is obtained which can be incorporated extremely well into cosmetic formulations without further effort, such as, for example, grinding or forced dispersion.

### Example 4

### Pigment Mixture Having a Red Mass Tone

### Batch 4A:

15 g of natural mica flakes from Merck KGaA (particle size<15 μm) together with 85 g of silicon dioxide spheres from ABC Nanotech (diameter 2-4 μm) are stirred into 1500 ml of deionized water. This suspension is heated to the reaction temperature of 75° C. with stirring. The pH is then set to pH 10 using 10% sodium hydroxide solution, kept there for 5 minutes and finally adjusted to the covering pH of 3.1 using 10% hydrochloric acid. 838 g of 14% iron chloride solution (170 g of Fe₂O₃/l) are subsequently metered in, during which the pH is kept constant using 20% sodium hydroxide solution. When the addition is complete, the mixture is stirred for a further 15 minutes. The pH is then set to pH 1.8 using 10% HCl, and 956 g of titanium tetrachloride solution (400 g/l, which corresponds to 133 g of TiO₂) are subsequently metered in. The pH is kept constant using 20% sodium hydroxide solution. When the addition is complete, the mixture is stirred for a further 5 minutes. The pH is then set to pH=5 using 10% sodium hydroxide solution, and the mixture is stirred for a further 15 minutes.

When this batch is complete, the suspension is filtered off, washed with deionized water, dried at 110° C. for 10 h and calcined at 800° C. for 30 min.

This precursor has a composition of 42% of Fe₂O₃, 33% of TiO₂, 21% of SiO₂ and 4% of mica.

### Batch 4B:

2 l of deionized water are heated to the reaction temperature of 85° C. with stirring, and the pH is then adjusted to 2.8 using 10% hydrochloric acid. 295 g of iron chloride solution (w(Fe)=14%) are subsequently metered in (corresponds to 60 g of Fe₂O₃), during which the pH is kept constant using 20% sodium hydroxide solution. When the addition is complete, the mixture is stirred for a further 5 minutes. The batch is not filtered off directly with suction.

60 g of the calcined material from batch 4A are subsequently added, and the pH is then set to pH=5 using 20% sodium hydroxide solution, and the mixture is stirred for a further 15 minutes. Finally, the suspension is filtered off, washed with 10 I of deionized water and dried at 110° C. for 10 h. Finally, the powder obtained is calcined at 800° C. for 30 min and sieved using a 24 μm sieve.

The final product has the following composition: 71% of Fe₂O₃, 16% of TiO₂, 11% of SiO₂ and 2% of mica.

A pigment mixture having a burgundy mass tone and a soft skin feel and matt texture is obtained which has a high chroma and can be incorporated extremely well into cosmetic formulations without further effort. The hiding power, skin feel and chroma can be adjusted as desired through the 4A/4B mixing ratio.

### Example 4a

If the 4A:4B ratio is 8:2, particularly highly chromatic pigment mixtures having an extraordinarily good skin feel and a moderate hiding power are obtained.

### Example 4b

If the 4A:4B ratio is 1:1, particularly highly chromatic pigment mixtures having a very good skin feel and a medium hiding power are obtained.

### Example 4c

If the 4A:4B ratio is 2:8, highly chromatic pigment mixtures having a very high hiding power and a good skin feel are obtained.

### Example 5

### Pigment Mixture Having a Champagne-Colored mass Tone

### Batch 5A:

8 g of aluminum oxide flakes from Merck KGaA (5-40 μm) together with 80 g of silicon dioxide spheres from Sinoenergy (diameter 2-4 μm) are stirred into 800 ml of deionized water. This suspension is heated to the reaction temperature of 73° C. with stirring, and the pH is then adjusted to 2.1 using 10% hydrochloric acid. 2200 g of a 25% titanium tetrachloride solution are subsequently metered in. The pH is kept constant using 20% sodium hydroxide solution. When the addition is complete, the mixture is stirred for a further 5 minutes. The pH is then set to pH 3.3 using 20% sodium hydroxide solution, and the mixture is stirred for a further 5 minutes. 356 g of a 7% iron chloride solution are subsequently metered in, during which the pH is kept constant using 20% sodium hydroxide solution. When the addition of the solution is complete, the mixture is stirred for a further minutes, and the pH is then set to pH 6 using 20% sodium hydroxide solution.

### Batch 5B:

1.7 l of deionized water are heated to the reaction temperature of 70° C. with stirring, and the pH is then adjusted to 2.1 using 10% hydrochloric acid. 1350 g of a 25% titanium tetrachloride solution are then metered in. The pH is kept constant using 20% sodium hydroxide solution. The pH is then adjusted to pH 3.3 using 20% sodium hydroxide solution, and the mixture is stirred for a further 5 minutes. The pH is subsequently set to 1.9, and 356 g of a 7% iron chloride solution are metered in, during which the pH is kept constant using 20% sodium hydroxide solution. When the addition is complete, the mixture is stirred for a further 5 minutes. The pH is then adjusted to pH 6 using 20% sodium hydroxide solution.

When both batches are complete, the suspensions of batches 5A and 5B are mixed, filtered off, washed and dried at 150° C. The pigment mixture is subsequently calcined at 780° C. and then passed through a sieve having a mesh width of 24 μm.

A champagne- to skin-colored pigment mixture having a soft skin feel and matt texture is obtained which has a high chroma and can be incorporated extremely well into cosmetic formulations without further effort. The hiding power, skin feel and chroma can be adjusted as desired through the 5A/5B mixing ratio.

### Example 5a

If the 5A:5B ratio is 8:2, particularly highly chromatic pigment mixtures having an extraordinarily good skin feel and a moderate hiding power are obtained.

### Example 5b

If the 5A:5B ratio is 1:1, particularly highly chromatic pigment mixtures having a very good skin feel and a medium hiding power are obtained.

Example 5c:

If the 5A:5B ratio is 2:8, highly chromatic pigment mixtures having a very high hiding power and a good skin feel are obtained.

### Example 6

### Pigment Mixture Having a Champagne-Colored Mass Tone

### Batch 6A:

12 g of natural mica flakes from Merck KGaA (<15 μm) together with 100 g of silicon dioxide spheres from ABC Nanotech (diameter 2-4 μm) are stirred into 800 ml of deionized water. This suspension is heated to the reaction temperature of 85° C. with stirring, and the pH is then adjusted to 2.1 using 10% hydrochloric acid. 1856 g of a 25% titanium tetrachloride solution are subsequently metered in. The pH is kept constant using 20% sodium hydroxide solution. When the addition is complete, the mixture is stirred for a further 5 minutes. The pH is then set to pH 3.3 using 20% sodium hydroxide solution, and the mixture is stirred for a further 5 minutes. 289 g of a 7% iron chloride solution are subsequently metered in, during which the pH is kept constant using 20% sodium hydroxide solution. When the addition of the solution is complete, the mixture is stirred for a further 5 minutes, and the pH is then set to pH 6 using 20% sodium hydroxide solution. The suspension is filtered, washed until salt-free and dried. The dried pigment mixture is calcined at 700° C. under air in a muffle furnace.

### Batch 6B:

1.7 l of deionized water are heated to the reaction temperature of 65° C. with stirring, and the pH is then adjusted to 2.1 using 10% hydrochloric acid. 1265 g of a 25% titanium tetrachloride solution are then metered in. The pH is kept constant using 20% sodium hydroxide solution. The pH is then adjusted to pH 3.1 using 20% sodium hydroxide solution, and the mixture is stirred for a further 5 minutes. The pH is subsequently set to 2.1, and 564 g of a 7% iron chloride solution are metered in, during which the pH is kept constant using 20% sodium hydroxide solution. When the addition is complete, the mixture is stirred for a further 5 minutes. The pH is then adjusted to pH 6 using 20% sodium hydroxide solution.

When both batches are complete, the calcined powder from batch 6A is added to the suspension of batch 6B. The suspension is filtered, washed and dried at 150° C. The powder is subsequently calcined at 780° C. and then passed through a sieve having a mesh width of 24 μm.

A champagne- to skin-colored pigment mixture having a soft skin feel and matt texture is obtained which has a high chroma and can be incorporated extremely well into cosmetic formulations without further effort. The hiding power, skin feel and chroma can be adjusted as desired through the 6A/6B mixing ratio.

### Example 6a

If the 6A:6B ratio is 8:2, particularly highly chromatic pigment mixtures having an extraordinarily good skin feel and a moderate hiding power are obtained.

### Example 6b

If the 6A:6B ratio is 1:1, particularly highly chromatic pigment mixtures having a very good skin feel and a medium hiding power are obtained.

### Example 6c

If the 6A:6B ratio is 2:8, highly chromatic pigment mixtures having a very good hiding power and a good skin feel are obtained.

### Example 7

### Pigment Mixture Having a Champagne-Colored Mass Tone

### Batch 7A

15 g of aluminum oxide flakes from Merck KGaA (<20 μm) together with 85 g of silicon dioxide spheres from ABC Nanotech (diameter 2-4 μm) are stirred into 800 ml of deionized water.

This suspension is heated to the reaction temperature of 73° C. with stirring, and the pH is then adjusted to 3.1 using 10% hydrochloric acid. 356 g of a 7% iron chloride solution are subsequently metered in, during which the pH is kept constant using 20% sodium hydroxide solution. When the addition is complete, the mixture is stirred for a further 5 minutes. The pH is then adjusted to pH 2.1 using 20% hydrochloric acid, and the mixture is stirred for a further 5 minutes. 1689 g of a 25% titanium tetrachloride solution are subsequently metered in. The pH is kept constant using 20% sodium hydroxide solution. When the addition of the solution is complete, the mixture is stirred for a further 5 minutes, and the pH is then set to pH 6 using 20% sodium hydroxide solution. The suspension is filtered, washed until salt-free and dried at 140° C.

### Batch 7B:

1.7 l of deionized water are heated to the reaction temperature of 85° C. with stirring, and the pH is then adjusted to 3.1 using 10% hydrochloric acid. 685 g of a 7% iron chloride solution are subsequently metered in, during which the pH is kept constant using 20% sodium hydroxide solution.

When the addition is complete, the mixture is stirred for a further 5 minutes. The pH is then adjusted to pH 2.1 using 10% hydrochloric acid, and 941 g of a 25% titanium tetrachloride solution are metered in. The pH is kept constant using 20% sodium hydroxide solution. The mixture is then stirred for a further 5 minutes, and the pH is adjusted to pH 5 using 20% sodium hydroxide solution.

When both batches are complete, the calcined powder from batch A is added to the suspension of batch B. The suspension is filtered, washed and dried at 150° C. The powder is subsequently calcined at 550° C. and then passed through a sieve having a mesh width of 24 μm.

A champagne- to skin-colored pigment mixture having a soft skin feel and matt texture is obtained which has a high chroma and can be incorporated extremely well into cosmetic formulations without further effort. The hiding power, skin feel and chroma can be adjusted as desired through the 7A/7B mixing ratio.

### Example 7a

If the 7A:7B ratio is 8:2, particularly highly chromatic pigment mixtures having an extraordinarily good skin feel and a moderate hiding power are obtained.

### Example 7b

If the 7A:7B ratio is 1:1, particularly highly chromatic pigment mixtures having a very good skin feel and a medium hiding power are obtained.

### Example 7c

If the 7A:7B ratio is 2:8, highly chromatic pigment having a very high hiding power and a good skin feel are obtained.

### Example 8

### Pigment Mixture Having a Black Mass Tone

### Batch 8A:

8 g of synthetic silicon dioxide flakes from Merck KGaA (5-50 μm) together with 80 g of silicon dioxide spheres from ABC Nanotech (diameter 2-4 μm are stirred into 800 ml of deionized water.

This suspension is heated to the reaction temperature of 73° C. with stirring, and the pH is then adjusted to 3.3 using 10% hydrochloric acid. 923 g of 10% iron chloride solution are subsequently metered in, during which the pH is kept constant using 20% sodium hydroxide solution. When the addition is complete, the mixture is stirred for a further 5 minutes. The pH is then set to pH 2.1 using 10% HCl, and 310 g of titanium tetrachloride solution (400 g/l) are subsequently metered in. During this, the pH is kept constant using 20% sodium hydroxide solution. When the addition is complete, the mixture is stirred for a further 5 minutes. The pH is then adjusted to pH 7 using 20% sodium hydroxide solution, and the mixture is stirred for a further 15 minutes.

### Batch 8B:

1.7 l of deionized water are heated to the reaction temperature of 73° C. with stirring, and the pH is then adjusted to 3.3 using 10% hydrochloric acid. 1000 g of 10% iron chloride solution are subsequently metered in, during which the pH is kept constant using 20% sodium hydroxide solution. When the addition is complete, the mixture is stirred for a further 5 minutes, and the pH is then set to pH 7 using 20% sodium hydroxide solution.

When both batches are complete, the suspensions of batches 8A and 8B are mixed, filtered off, washed and dried at 150° C. The powder is subsequently calcined at 650° C. under forming gas (N₂:H₂ ratio 95:5) and then passed through a sieve having a mesh width of 24 μm.

An effect pigment having a black mass tone and a soft skin feel and matt texture is obtained which has a high chroma and can be incorporated extremely well into cosmetic formulations without further effort. The hiding power, skin feel and chroma can be adjusted as desired through the 8A/8B mixing ratio.

### Example 8a

If the 8A:8B ratio is 8:2, particularly highly chromatic pigment mixtures having an extraordinarily good skin feel and a moderate hiding power are obtained.

### Example 8b

If the 8A:8B ratio is 1:1, particularly highly chromatic pigment mixtures having a very good skin feel and a medium hiding power are obtained.

### Example 8c

If the 8A:8B ratio is 2:8, highly chromatic pigment mixtures having a very high hiding power and a good skin feel are obtained.

### Example 9

### Pigment Mixture Having a Black Mass Tone

### Batch 9A

15 g of natural mica flakes from Merck KGaA (<15 μm) together with 85 g of silicon dioxide spheres from ABC Nanotech (diameter 2-4 μm) are stirred into 1200 ml of deionized water.

This suspension is heated to the reaction temperature of 80° C. with stirring, and the pH is then adjusted to 3.1 using 10% hydrochloric acid. 886 g of 14% iron chloride solution are subsequently metered in, during which the pH is kept constant using 32% sodium hydroxide solution. When the addition is complete, the mixture is stirred for a further 5 minutes. The pH is then adjusted to pH 2.1 using 10% HCl, and 1240 g of titanium tetrachloride solution (400 g/I) are subsequently metered in. The pH is kept constant using 20% sodium hydroxide solution. When the addition is complete, the mixture is stirred for a further 5 minutes. The pH is then set to pH 7 using 20% sodium hydroxide solution, and the mixture is stirred for a further 15 minutes. The suspension is filtered, washed until salt-free and dried. The dried powder is calcined at 800° C. under air in a muffle furnace.

### Batch 9B:

1.7 l of deionized water are heated to the reaction temperature of 82° C. with stirring, and the pH is then adjusted to 3.2 using 10% hydrochloric acid. 495 g of 14% iron chloride solution are subsequently metered in, during which the pH is kept constant using 20% sodium hydroxide solution. When the addition is complete, the mixture is stirred for a further 5 minutes, and the pH is then set to pH 5 using 20% sodium hydroxide solution.

When both batches are complete, the calcined powder from batch A is added to the suspension of batch B. The suspension is filtered, washed and dried at 150° C. The combined powder is subsequently calcined at 500° C. under forming gas (N₂:H₂ ratio =92:8) and then passed through a sieve having a mesh width of 24 μm.

An effect pigment having a black mass tone and a soft skin feel and matt texture is obtained which has a high chroma and can be incorporated extremely well into cosmetic formulations without further effort. The hiding power, skin feel and chroma can be adjusted as desired through the 9A/9B mixing ratio.

### Example 9a:

If the 9A:9B ratio is 8:2, particularly highly chromatic pigment mixtures having an extraordinarily good skin feel and a moderate hiding power are obtained.

### Example 9b

If the 9A:9B ratio is 1:1, particularly highly chromatic pigment mixtures having a very good skin feel and a medium hiding power are obtained.

### Example 9c

If the 9A:9B ratio is 2:8, highly chromatic pigment mixtures having a very good hiding power and a good skin feel are obtained.

### Example 10

### Pigment Mixture Having a Black Mass Tone

### Batch 10A:

10 g of natural mica flakes from Merck KGaA (10-60 μm) together with 85 g of silicon dioxide spheres from Sinoenergy (diameter 2-4 μm) are stirred into 1200 ml of deionized water.

This suspension is heated to the reaction temperature of 85° C. with stirring, and the pH is then adjusted to 2.9 using 10% hydrochloric acid. 925 g of 14% iron chloride solution are subsequently metered in, during which the pH is kept constant using 32% sodium hydroxide solution. When the addition is complete, the mixture is stirred for a further 5 minutes. The pH is then set to pH 2.1 using 10% HCl, and 1015 g of titanium tetrachloride solution (400 g/l) are subsequently metered in. During this, the pH is kept constant using 20% sodium hydroxide solution. When the addition is complete, the mixture is stirred for a further 5 minutes. The pH is then adjusted to pH 7 using 20% sodium hydroxide solution, and the mixture is stirred for a further 15 minutes. The suspension is filtered, washed until salt-free and dried.

### Batch 10B:

1.7 l of deionized water are heated to the reaction temperature of 70° C. with stirring, and the pH is then adjusted to 3.2 using 10% hydrochloric acid. 750 g of 14% iron chloride solution are subsequently metered in, during which the pH is kept constant using 20% sodium hydroxide solution. When the addition is complete, the mixture is stirred for a further 5 minutes, and the pH is then set to pH 5 using 20% sodium hydroxide solution.

When both batches are complete, the dried powder from batch A is added to the suspension of batch B. The suspension is filtered, washed and dried at 150° C. The combined powder is subsequently calcined at 760° C. under forming gas (N₂:H₂ ratio =92:8) and passed through a sieve having a mesh width of 24 μm.

An effect pigment having a black mass tone and a soft skin feel and matt texture is obtained which has a high chroma and can be incorporated extremely well into cosmetic formulations without further effort. The hiding power, skin feel and chroma can be adjusted as desired through the 10A/10B mixing ratio.

### Example 10a

If the 10A:10B ratio is 8:2, particularly highly chromatic pigment mixtures having an extraordinarily good skin feel and a moderate hiding power are obtained.

### Example 10b

If the 10A:10B ratio is 1:1, particularly highly chromatic pigment mixtures having a very good skin feel and a medium hiding power are obtained.

### Example 10c

If the 10A:10B ratio is 2:8, highly chromatic pigment mixtures having a very high hiding power and a good skin feel are obtained.

### Example 11

### Pigment Mixture Having a Red Mass Tone

### Batch 11A:

15 g of natural mica flakes from Merck KGaA (5-35 μm) together with 85 g of silicon dioxide spheres from ABC Nanotech (diameter 2-4 μm) are stirred into 1200 ml of deionized water.

This suspension is heated to the reaction temperature of 80° C. with stirring, and the pH is then adjusted to 3.1 using 10% hydrochloric acid. 886 g of 14% iron chloride solution are subsequently metered in, during which the pH is kept constant using 32% sodium hydroxide solution. When the addition is complete, the mixture is stirred for a further 5 minutes. The pH is then set to 2.1 using 10% HCl, and 1240 g of titanium tetrachloride solution (400 g/l) are subsequently metered in. During this, the pH is kept constant using 20% sodium hydroxide solution. When the addition is complete, the mixture is stirred for a further 5 minutes. The pH is then adjusted to pH 7 using 20% sodium hydroxide solution, and the mixture is stirred for a further 15 minutes. The suspension is filtered off, washed until salt-free and dried. The dried powder is calcined at 800° C. in a muffle furnace for 30 minutes.

### Batch 11B:

1.7 l of deionized water are heated to the reaction temperature of 82° C. with stirring, and the pH is then adjusted to 3.2 using 10% hydrochloric acid. 495 g of 14% iron chloride solution are subsequently metered in, during which the pH is kept constant using 20% sodium hydroxide solution. When the addition is complete, the mixture is stirred for a further 5 minutes, and the pH is then adjusted to pH 5 using 20% sodium hydroxide solution.

When both batches are complete, the calcined powder from batch 11A is added to the suspension of batch 11B. The suspension is filtered, washed and dried at 150° C. The combined powder is subsequently calcined at 780° C. and then passed through a sieve having a mesh width of 24 μm.

An effect pigment having a burgundy mass tone and a soft skin feel and matt texture is obtained which has a high chroma and can be incorporated extremely well into cosmetic formulations without further effort. The hiding power, skin feel and chroma can be adjusted as desired through the 11A:11B mixing ratio.

### Example 11 a

If the 11A:11 B ratio is 8:2, particularly highly chromatic pigment mixtures having an extraordinarily good skin feel and a moderate hiding power are obtained.

Example 11 b:

If the 11A:11 B ratio is 1:1, particularly highly chromatic pigment mixtures having a very good skin feel and a medium hiding power are obtained.

### Example 11 c

If the 11A:11 B ratio is 2:8, highly chromatic pigment mixtures having a very high hiding power and a good skin feel are obtained.

### Example 12

### Pigment Mixture Having A Red Mass Tone

### Batch 12A:

10 g of synthetic mica flakes from Merck KGaA (<15 μm) together with 85 g of silicon dioxide spheres from Sinoenergy (diameter 2-4 μm) are stirred into 1200 ml of deionized water.

This suspension is heated to the reaction temperature of 85° C. with stirring, and the pH is then adjusted to 2.9 using 10% hydrochloric acid. 925 g of 14% iron chloride solution are subsequently metered in, during which the pH is kept constant using 32% sodium hydroxide solution. When the addition is complete, the mixture is stirred for a further 5 minutes. The pH is then set to pH 2.1 using 10% HCl, and 1015 g of titanium tetrachloride solution (400 g/l) are subsequently metered in. During this, the pH is kept constant using 20% sodium hydroxide solution. When the addition is complete, the mixture is stirred for a further 5 minutes. The pH is then adjusted to pH 7 using 20% sodium hydroxide solution, and the mixture is stirred for a further 15 minutes. The suspension is filtered off, washed until salt-free and dried.

### Batch 12B:

1.7 l of deionized water are heated to the reaction temperature of 70° C. with stirring, and the pH is then adjusted to 3.2 using 10% hydrochloric acid. 750 g of 14% iron chloride solution are subsequently metered in, during which the pH is kept constant using 20% sodium hydroxide solution. When the addition is complete, the mixture is stirred for a further 5 minutes, and the pH is then set to pH 5 using 20% sodium hydroxide solution.

When both batches are complete, the dried powder from batch 12A is added to the suspension of batch 12B. The suspension is filtered, washed and dried at 150° C. The combined powder is subsequently calcined at 780° C. and passed through a sieve having a mesh width of 24 μm.

A pigment mixture having a burgundy mass tone and a soft skin feel and matt texture is obtained which has a high chroma and can be incorporated extremely well into cosmetic formulations without further effort. The hiding power, skin feel and chroma can be adjusted as desired through the 12A/12B mixing ratio.

### Example 12a

If the 12A:12B ratio is 8:2, particularly highly chromatic pigment mixtures having an extraordinarily good skin feel and a moderate hiding power are obtained.

### Example 12b

If the 12A:12B ratio is 1:1, particularly highly chromatic pigment mixtures having a very good skin feel and a medium hiding power are obtained.

### Example 12c

If the 12A:12B ratio is 2:8, highly chromatic pigment mixtures having a very good hiding power and a good skin feel are obtained.

### Example 13

### Pigment Mixture Having a Yellow Mass Tone

### Batch 13A:

8 g of synthetic mica flakes from Merck KGaA (10-40 μm) together with 80 g of silicon dioxide spheres from ABC Nanotech (diameter 2-4 μm) are stirred into 800 ml of deionized water.

This suspension is heated to the reaction temperature of 85° C. with stirring, and the pH is then adjusted to 3.5 using 10% sulfuric acid. A solution consisting of 101 g of iron(III) sulfate, 130 g of iron(II) sulfate heptahydrate and 480 g of deionized water is subsequently metered in, during which the pH is kept constant using 20% sodium hydroxide solution. When the addition is complete, the mixture is stirred for a further 5 minutes. The pH is then set to pH 8 using 20% sodium hydroxide solution, after which 150 g of a sodium water-glass solution (w SiO₂=14%) is metered in. During this, the pH is kept constant using 20% sulfuric acid. When the addition is complete, the mixture is stirred for a further 5 minutes. The pH is then set to pH 5 using 10% sulfuric acid, and the mixture is stirred for a further 15 min.

### Batch 13B:

1.7 l of deionized water are heated to the reaction temperature of 87° C. with stirring, and the pH is then adjusted to 3.7 using 10% hydrochloric acid. A solution consisting of 190 g of iron(III) sulfate, 210 g of iron(II) sulfate heptahydrate and 600 g of deionized water is subsequently metered in, during which the pH is kept constant using 20% sodium hydroxide solution. When the addition is complete, the mixture is stirred for a further 5 minutes, and the pH is then adjusted to pH 7 using 20% sodium hydroxide solution, after which 90 g of a sodium water-glass solution (w SiO₂ =14%) is metered in. During this, the pH is kept constant using 20% sulfuric acid. When the addition is complete, the mixture is stirred for a further 5 minutes. The pH is then set to pH 5 using 10% sulfuric acid, and the mixture is stirred for a further 15 min.

When both batches are complete, the suspensions of batches 13A and 13B are mixed, filtered off, washed and dried at 150° C. and passed through a sieve having a mesh width of 24 μm.

A pigment mixture having a yellow mass tone and a soft skin feel and matt texture is obtained which has a high chroma and can be incorporated extremely well into cosmetic formulations without further effort. The hiding power, skin feel and chroma can be adjusted as desired through the 13A/13B mixing ratio.

### Example 13a

If the 13A:13B ratio is 8:2, particularly highly chromatic pigment mixtures having an extraordinarily good skin feel and a moderate hiding power are obtained.

### Example 13b

If the 13A:13B ratio is 1:1, particularly highly chromatic pigment mixtures having a very good skin feel and a medium hiding power are obtained.

### Example 13c

If the 13A:13B ratio is 2:8, highly chromatic pigment mixtures having a very good hiding power and a good skin feel are obtained.

### Example 14

### Pigment Mixture Having a Yellow Mass Tone

### Batch 14A:

15 g of synthetic mica flakes Merck KGaA (5-40 μm) together with 85 g of silicon dioxide spheres from Sinoenergy (diameter 2-4 μm) are stirred into 1200 ml of deionized water.

This suspension is heated to the reaction temperature of 90° C. with stirring, and the pH is then adjusted to 3.5 using 10% sulfuric acid. A solution consisting of 200 g of iron(III) sulfate, 210 g of iron(II) sulfate heptahydrate and 700 g of deionized water is subsequently metered in, during which the pH is kept constant using 20% sodium hydroxide solution. When the addition is complete, the mixture is stirred for a further 5 minutes. The pH is then set to pH 8 using 20% sodium hydroxide solution, after which 290 g of a sodium water-glass solution (w SiO₂=14%) is metered in. During this, the pH is kept constant using 20% sulfuric acid. When the addition is complete, the mixture is stirred for a further 5 minutes. The pH is then set to pH 5 using 10% sulfuric acid, and the mixture is stirred for a further 15 min. The suspension is filtered, washed until salt-free and dried.

### Batch 14B:

1.2 l of deionized water are heated to the reaction temperature of 75° C. with stirring, and the pH is then adjusted to 3.9 using 10% hydrochloric acid. A solution consisting of 120 g of iron(III) sulfate, 163 g of iron(II) sulfate heptahydrate and 500 g of deionized water is subsequently metered in, during which the pH is kept constant using 20% sodium hydroxide solution. When the addition is complete, the mixture is stirred for a further 5 minutes, and the pH is then adjusted to pH 7 using 20% sodium hydroxide solution, after which 65 g of a sodium water-glass solution (w SiO₂=14%) is metered in. During this, the pH is kept constant using 20% sulfuric acid.

When the addition is complete, the mixture is stirred for a further 5 minutes. The pH is then set to pH 6 using 10% sulfuric acid, and the mixture is stirred for a further 15 min.

When both batches are complete, the dried powder from batch 14A is added to the suspension of batch 14B. The suspension is filtered, washed and dried at 170° C. and passed through a sieve having a mesh width of 24 μm.

A pigment mixture having a yellow mass tone and a soft skin feel and matt texture is obtained which has a high chroma and can be incorporated extremely well into cosmetic formulations without further effort. The hiding power, skin feel and chroma can be adjusted as desired through the 14A/14B mixing ratio.

### Example 14a

If the 14A:14B ratio is 8:2, particularly highly chromatic pigment mixtures having an extraordinarily good skin feel and a moderate hiding power are obtained.

### Example 14b

If the 14A:14B ratio is 1:1, particularly highly chromatic pigment mixtures having a very good skin feel and a medium hiding power are obtained.

### Example 14c

If the 14A:14B ratio is 2:8, highly chromatic pigment mixtures having a very good hiding power and a good skin feel are obtained.

### Example 15

### Pigment Mixture Having a Green Mass Tone

### Batch 15A

8 g of natural mica flakes from Merck KGaA (particle size 10-100 μm) together with 80 g of silicon dioxide spheres from ABC Nanotech (diameter 2-4 μm) are stirred into 1200 ml of deionized water.

This suspension is heated to the reaction temperature of 70° C. with stirring, and the pH is then adjusted to 5.5 using 10% hydrochloric acid. A solution of 309 g of chromium(III) chloride hexahydrate and 800 ml of deionized water is subsequently metered in, during which the pH is kept constant using 20% sodium hydroxide solution. When the addition is complete, the mixture is stirred for a further 5 minutes. The pH is then set to pH 7 using 20% sodium hydroxide solution, and the mixture is stirred for a further 15 min.

### Batch 15B:

2.1 l of deionized water are heated to the reaction temperature of 73° C. with stirring, and the pH is then adjusted to 6.0 using 10% hydrochloric acid. A solution of 556 g of chromium(III) chloride hexahydrate and 1250 ml of deionized water is subsequently metered in, during which the pH is kept constant using 20% sodium hydroxide solution. When the addition is complete, the mixture is stirred for a further 5 minutes, and the pH is then set to pH 7 using 20% sodium hydroxide solution.

When both batches are complete, the suspensions of batches 15A and 15B are mixed, filtered off, washed and dried at 150° C. The powder is subsequently calcined at 800° C. under air and then sieved through a sieve having a mesh width of 24 μm.

A pigment mixture having a green mass tone and a soft skin feel and matt texture is obtained which has a high chroma and can be incorporated extremely well into cosmetic formulations without further effort. The hiding power, skin feel and chroma can be adjusted as desired through the 15A/15B mixing ratio.

### Example 15a

If the 15A:15B ratio is 8:2, particularly highly chromatic pigment mixtures having an extraordinarily good skin feel and a moderate hiding power are obtained.

### Example 15b

If the 15A:15B ratio is 1:1, particularly highly chromatic pigment mixtures having a very good skin feel and a medium hiding power are obtained.

### Example 15c

If the 15A:15B ratio is 2:8, highly chromatic pigment mixtures having a very high hiding power and a good skin feel are obtained.

### Example 16

### Pigment Mixture Having a Green Mass Tone

### Batch 16A:

15 g of natural mica flakes from Merck KGaA (particle size 5-25 μm) together with 85 g of silicon dioxide spheres from Sinoenergy (diameter 2-4 μm) are stirred into 1800 ml of deionized water.

This suspension is heated to the reaction temperature of 80° C. with stirring, and the pH is then adjusted to 1.8 using 10% hydrochloric acid. 350 g of titanium tetrachloride solution (400 g/l) are subsequently metered in. The pH is kept constant using 20% sodium hydroxide solution. When the addition is complete, the mixture is stirred for a further 5 minutes. The pH is then set to pH 7 using 20% sodium hydroxide solution, and the mixture is stirred for a further 15 min. A solution of 516 g of chromium(III) chloride hexahydrate and 1010 ml of deionized water is subsequently metered in, during which the pH is kept constant using 20% sodium hydroxide solution. When the addition is complete, the mixture is stirred for a further 5 minutes. The suspension is filtered, washed until salt-free and dried. The dried powder is calcined at 800° C. under air in a muffle furnace.

### Batch 16B:

1.9 l of deionized water are heated to the reaction temperature of 80° C. with stirring, and the pH is then adjusted to 6.0 using 10% hydrochloric acid. A solution of 706 g of chromium(III) chloride hexahydrate and 1200 ml of deionized water is subsequently metered in, during which the pH is kept constant using 20% sodium hydroxide solution. When the addition is complete, the mixture is stirred for a further 5 minutes, and the pH is then set to pH 7 using 20% sodium hydroxide solution.

When both batches are complete, the calcined powder from batch A is added to the suspension of batch B. The suspension is filtered, washed and dried at 150° C. The combined powder is subsequently calcined at 750° C. under air and passed through a sieve having a mesh width of 24 μm.

A pigment mixture having a green mass tone and a soft skin feel and matt texture is obtained which has a high chroma and can be incorporated extremely well into cosmetic formulations without further effort. The hiding power, skin feel and chroma can be adjusted as desired through the 16A/16B mixing ratio.

### Example 16a:

If the 16A:16B ratio is 8:2, particularly highly chromatic pigment mixtures having an extraordinarily good skin feel and a moderate hiding power are obtained.

### Example 16b:

If the 16A:16B ratio is 1:1, particularly highly chromatic pigment mixtures having a very good skin feel and a medium hiding power are obtained.

### Example 16c:

If the 16A:16B ratio is 2:8, highly chromatic pigment mixtures having a very high hiding power and a good skin feel are obtained.

### Example 17

### Pigment Mixture Having a Green Mass Tone

### Batch 17A:

10 g of natural mica flakes from Merck KGaA (particle size<15 μm) together with 100 g of silicon dioxide spheres from ABC Nanotech (diameter 2-4 μm)) are stirred into 1800 ml of deionized water.

This suspension is heated to the reaction temperature of 80° C. with stirring, and the pH is then adjusted to 2.1 using 10% hydrochloric acid. 200 g of titanium tetrachloride solution (400 g/l) are subsequently metered in. The pH is kept constant using 20% sodium hydroxide solution. When the addition is complete, the mixture is stirred for a further 5 minutes. The pH is then set to pH 7.5 using 20% sodium hydroxide solution, and the mixture is stirred for a further 15 min. A solution of 480 g of chromium(III) chloride hexahydrate and 900 ml of deionized water is subsequently metered in, during which the pH is kept constant using 20% sodium hydroxide solution. When the addition is complete, the mixture is stirred for a further 5 minutes. The suspension is filtered, washed until salt-free and dried.

### Batch 17B:

1.9 I of deionized water are heated to the reaction temperature of 85° C. with stirring, and the pH is then adjusted to 6.5 using 10% hydrochloric acid. A solution of 706 g of chromium(III) chloride hexahydrate and 950 ml of deionized water is subsequently metered in, during which the pH is kept constant using 20% sodium hydroxide solution. When the addition is complete, the mixture is stirred for a further 5 minutes, and the pH is then adjusted to pH 7 using 20% sodium hydroxide solution.

When both batches are complete, the dried powder from batch 17A is added to the suspension of batch 17B. The suspension is filtered, washed and dried at 150° C. The combined powder is subsequently calcined at 850° C. under air and passed through a sieve having a mesh width of 24 μm.

A pigment mixture having a green mass tone and a soft skin feel and matt texture is obtained which has a high chroma and can be incorporated extremely well into cosmetic formulations without further effort. The hiding power, skin feel and chroma can be adjusted as desired through the 17A/17B mixing ratio.

### Example 17a

If the 17A:17B ratio is 8:2, particularly highly chromatic pigment mixtures having an extraordinarily good skin feel and a moderate hiding power are obtained.

### Example 17b

If the 17A:17B ratio is 1:1, particularly highly chromatic pigment mixtures having a very good skin feel and a medium hiding power are obtained.

### Example 17c

If the 17A:17B ratio is 2:8, highly chromatic pigment mixtures having a very high hiding power and a good skin feel are obtained.

### Example 18

### Pigment Mixture Having a White Mass Tone

### Batch 18A

20 g of natural mica flakes from Merck KGaA (particle size 10-60 μm) together with 65 g of silicon dioxide spheres from Sinoenergy (diameter 2-4 μm) are stirred into 1200 ml of deionized water.

This suspension is heated to the reaction temperature of 75° C. with stirring, and the pH is then adjusted to 2.0 using 10% hydrochloric acid. 150 g of 10% tin(IV) chloride solution are subsequently metered in over the course of 45 min, during which the pH is kept constant using 20% sodium hydroxide solution. When the addition is complete, the mixture is stirred for a further 5 minutes. 1300 g of 12% titanium tetrachloride solution are then metered in over the course of 6 hours. The pH is kept constant using 20% sodium hydroxide solution. When the addition is complete, the mixture is stirred for a further 5 minutes. The pH is then set to pH 6 using 20% sodium hydroxide solution, and the mixture is stirred for a further 15 min.

### Batch 18B:

1.5 l of deionized water are heated to the reaction temperature of 75° C. with stirring, and the pH is then adjusted to pH 3.0 using 10% hydrochloric acid. 1900 g of 14% titanium tetrachloride solution are subsequently metered in over the course of 4 hours, during which the pH is kept constant using 20% sodium hydroxide solution. When the addition is complete, the mixture is stirred for a further 5 minutes, and the pH is then set to pH 7 using 20% sodium hydroxide solution.

When both batches are complete, the suspensions of batches 18A and 18B are mixed, filtered off, washed with water and dried at 150° C. The powder is subsequently calcined at 800° C. for 45 min and then passed through a sieve having a mesh width of 100 μm.

A pigment mixture having a white mass tone, a soft skin feel, a matt texture and an elegant shimmer is obtained which can be incorporated extremely well into cosmetic formulations without further effort, such as, for example, grinding or forced dispersion. The hiding power, skin feel and luminance (shimmer) can be adjusted as desired through the 18A/18B mixing ratio.

### Example 18a

If the 18A:18B ratio is 8:2, particularly highly chromatic pigment mixtures having an extraordinarily good skin feel and a moderate hiding power are obtained.

### Example 18b

If the 18A:18B ratio is 1:1, particularly highly chromatic pigment mixtures having a very good skin feel and a medium hiding power are obtained.

### Example 18c

If the 18A:18B ratio is 2:8, highly chromatic pigment mixtures having a very high hiding power and a good skin feel are obtained.

### Example 19

### Pigment Mixture Having a Blue Mass Tone

### Batch 19A:

8 g of glass flakes from Merck KGaA (particle size 10-100 μm together with 80 g of silicon dioxide spheres from ABC Nanotech (diameter: 2-4 μm)) are stirred into 800 ml of deionized water.

This suspension is heated to the reaction temperature of 75° C. with stirring. The pH is adjusted to 2.1 by means of 10% sulfuric acid, and 2200 g of a 25% titanium tetrachloride solution are then metered in. The pH is kept constant using 20% sodium hydroxide solution. When the addition is complete, the mixture is stirred for a further 5 minutes, and the pH is then adjusted to 4.3 using 10% sulfuric acid. Two solutions 1 and 2 are subsequently added simultaneously, while the pH is kept constant using a 10% ammonium carbonate solution. Solution 1 is prepared from 60 g of potassium hexacyanoferrate(III) and 1300 g of deionized water. Solution 2 consists of 76 g of iron sulfate heptahydrate and 1250 g of deionized water. When the addition is complete, the mixture is stirred for a further 15 min, and the pH is then set to pH 6 using 20% sodium hydroxide solution.

### Batch 19B:

900 ml of deionized water are heated to the reaction temperature of 73° C. with stirring, and the pH is then adjusted to 4.3 using 10% sulfuric acid. Two solutions 1 and 2 are subsequently added simultaneously, while the pH is kept constant using a 10% ammonium carbonate solution. Solution 1 is prepared from 165 g of potassium hexacyanoferrate(III) and 2100 g of deionized water. Solution 2 consists of 209 g of iron sulfate heptahydrate and 1950 g of deionized water. The pH is then set to pH 6 using 20% sodium hydroxide solution.

When both batches are complete, the suspensions of batches 1A and 1B are mixed, filtered off, washed and dried at 150° C. and then passed through a sieve having a mesh width of 24 μm.

A pigment mixture having a blue mass tone and a soft skin feel and matt texture is obtained which has a high chroma and can be incorporated extremely well into cosmetic formulations without further effort. The hiding power, skin feel and chroma can be adjusted as desired through the 19A/19B mixing ratio.

### Example 19a

If the 19A:19B ratio is 8:2, particularly highly chromatic pigment mixtures having an extraordinarily good skin feel and a moderate hiding power are obtained.

### Example 19b

If the 19A:19B ratio is 1:1, particularly highly chromatic pigment mixtures having a very good skin feel and a medium hiding power are obtained.

### Example 19c

If the 19A:19B ratio is 2:8, highly chromatic pigment mixtures having a very high hiding power and a good skin feel are obtained.

### Example 20

### Pigment Mixture Having a White Mass Tone

1500 ml of deionized water are initially introduced in a heatable reaction vessel and heated to 75° C., and 376 g of TiOCl₂ solution (400 g of TiCl₄/liter) are metered in at this temperature at a metering rate of 5 ml/min. with vigorous stirring. The pH drops in the process and is kept constant at 2.0 by controlled addition of 32% sodium hydroxide solution. When the addition of TiOCl₂ is complete, the mixture is stirred at pH 2.0 for 15 min. A homogeneous suspension of 75 g of mica flakes (particle size 1 - 15 pm) and 25 g of SiO₂ spheres (D₅₀=2−4 μm; SILNOS 130 from ABC Nanotech Co., Ltd.) in 250 ml of deionized water is subsequently added, and, when 75° C. is re-attained, the pH is lowered to 2.2 using hydrochloric acid (18% HCl). While keeping the temperature and pH constant using 32% sodium hydroxide solution, a homogeneous solution of 8,64 g of SnOCl₂ solution (50% SnCl₄), 22.8 g of hydrochloric acid (37% HCl) and 167 g of deionized water is metered in at a uniform rate over the course of 30 min. After a subsequent stirring time of 15 min., 740 g of TiOCl₂ solution (400 g of TiCl₄/liter) are added at a metering rate of 5 ml/min. During this, the pH is kept constant at 2.2 using 32% sodium hydroxide solution. When the addition of the TiOCl₂ solution is complete, the mixture is stirred for a further 15 min., the pH is then adjusted to pH 5.0 using 32% sodium hydroxide solution, and the mixture is stirred for a further 15 min.

The solid obtained is filtered off, washed with 15 l of deionized water and dried at 110° C. for 16 hours. The product is subsequently calcined at 850° C. for 30 min. and sieved through a sieve having a mesh width of 100 μm.

A white powder having a very soft skin feel is obtained which exhibits a slight bluish luster when spread on the skin.

### Use Examples

### Example A1

### Eye Shadow Gel

| Raw material | INCI | [%] |
|---|---|---|
| Phase A | | |
| Timiron ® Super | (1) MICA FLAKES FROM MERCK | 15.00 |
| Gold | KGAA, CI 77891 (TITANIUM | |
| | DIOXIDE) | |
| Pigment mixture | | 7.00 |
| from Example 1 | | |
| Carbopol Ultrez | (2) ACRYLATES/C10-30 ALKYL | 0.30 |
| 21 | ACRYLATE CROSSPOLYMER | |
| Aloe vera powder | (3) ALOE BARBADENSIS | 0.05 |
| regular 200x | | |
| Citric acid | (1) CITRIC ACID | 0.00 |
| monohydrate | | |
| Water, | AQUA (WATER) | 55.87 |
| demineralized | | |
| Phase B | | |
| Triethanolamine | (4) TRIETHANOLAMINE, AQUA | 0.78 |
| 90% Care | (WATER) | |
| Germaben II | (5) PROPYLENE GLYCOL, | 1.00 |
| | DIAZOLIDINYL UREA, | |
| | METHYLPARABEN, | |
| | PROPYLPARABEN | |
| Glycerin, | (1) GLYCERIN | 2.00 |
| anhydrous | | |
| Water, | AQUA (WATER) | 13.00 |
| demineralized | | |
| Phase C | | |
| Lubrajel DV | (6) PROPYLENE GLYCOL, | 5.00 |
| | POLYGLYCERYL | |
| | METHACRYLATE | |

### Preparation:

Dissolve the aloe vera powder in the water of phase A, then add all pigments and the pigment mixture and the remaining ingredients apart from the Carbopol and disperse. Acidify using a few drops of citric acid in order to reduce the viscosity, then scatter in the Carbopol with stirring. When completely dissolved, slowly stir in the pre-dissolved phase B (do not homogenize), and subsequently add phase C. If necessary, adjust the pH to between 7.0-7.5 using citric acid solution.

A water-based eye shadow gel formulation containing aloe vera is obtained (extremely fast-drying and easy to apply using the fingers).

### Sources of Supply:

(1) Merck KGaA/Rona®
(2) Noveon
(3) Terry Laboratoires, Inc.
(4) BASF AG
(5) ISP Global Technologies
(6) Guardian

### Example A2

### Creamy Eye Shadow

| Raw material | INCI | [%] |
|---|---|---|
| Phase A | | |
| Colorona ® Light | (1) MICA FLAKES FROM MERCK | 10.00 |
| Blue | KGAA, CI 77891 (TITANIUM | |
| | DIOXIDE) | |
| | CI 77510 (FERRIC | |
| | FERROCYANIDE) | |
| Pigment mixture | | 15.00 |
| from Example 1 | | |
| Talc | (1) TALC | 12.00 |
| Phase B | | |
| Crodamol PMP | (2) PPG-2 MYRISTYL ETHER | 32.80 |
| | PROPIONATE | |
| Miglyol 812 N | (3) CAPRYLIC/CAPRIC | 12.00 |
| | TRIGLYCERIDE | |
| Syncrowax HGLC | (2) C18-36 ACID TRIGLYCERIDE | 10.00 |
| Syncrowax HRC | (2) TRIBEHENIN | 3.00 |
| Parteck ® LUB STA | (1) STEARIC ACID | 3.00 |
| Antaron V-216 | (4) PVP/HEXADECENE | 2.00 |
| | COPOLYMER | |
| Oxynex ® K liquid | (1) PEG-8, TOCOPHEROL, | 0.10 |
| | ASCORBYL PALMITATE, | |
| | ASCORBIC ACID, CITRIC | |
| | ACID | |
| Propyl 4-hydroxy- | (1) PROPYLPARABEN | 0.10 |
| benzoate | | |

### Preparation:

Heat phase B to about 80° C. until everything has melted and cool to 65° C. with stirring. Then add the ingredients of phase A with stirring, and pour the composition into the packaging provided at 65° C. Allow to cool to room temperature.

### Sources of Supply:

(1) Merck KGaA/Rona®
(2) Croda GmbH
(3) Sasol Germany GmbH
(4) ISP Global Technologies

### Example A3

### Face Powder

| Raw material | INCI | [%] |
|---|---|---|
| Phase A | | |
| Pigment mixture | | 20.00 |
| from Example 5 | | |
| Unipure Yellow LC | (1) CI 77492 (IRON OXIDES) | 1.20 |
| 182 | | |
| Unipure Red LC | (1) CI 77491 (IRON OXIDES) | 0.20 |
| 381 | | |
| Unipure Brown LC | (1) CI 77491 (IRON OXIDES) | 0.30 |
| 889 | CI 77499 (IRON OXIDES) | |
| Magnesium | (2) MAGNESIUM STEARATE | 2.00 |
| stearate | | |
| Talc | (2) TALC | 71.90 |
| Phase B | | |
| RonaCare ® | (2) TOCOPHERYL ACETATE | 0.30 |
| all-rac-alpha- | | |
| tocopheryl acetate | | |
| Perfume oil 200 | (3) PERFUME | 0.30 |
| 529 | | |
| Eutanol G | (4) OCTYLDODECANOL | 3.70 |
| Propyl 4-hydroxy- | (2) PROPYLPARABEN | 0.10 |
| benzoate | | |

### Preparation:

Add the constituents of phase A to the mixer (for example La Moulinette from Moulinex) and mix for 2×10 seconds. Transfer the mixture into a beaker, add phase B, and stir in advance using the spatula. Again add the mixture of phase A and phase B to the mixer and process for 3×10 seconds to give a homogeneous phase.

The pressing pressure for a powder tray having a diameter of 36 mm is about 25 bar.

### Sources of Supply:

(1) Les Colorants Wackherr
(2) Merck KGaA/Rona®
(3) Fragrance Resources
(4) Cognis GmbH

### Example A4

### Mattifying Foundation

| Raw material | INCI | [%] |
|---|---|---|
| Phase A | | |
| Water, | AQUA (WATER) | 57.89 |
| demineralized | | |
| Pigment mixture | | 6.00 |
| from Example 13 | | |
| Glycerin (87% | (1) GLYCERIN, AQUA (WATER) | 5.00 |
| extra pure) | | |
| RonaCare ® ectoin | (1) ECTOIN | 0.30 |
| Keltrol CG-SFT | (2) XANTHAN GUM | 0.15 |
| Triethanolamine | (3) TRIETHANOLAMINE, AQUA | 0.13 |
| 90% Care | (WATER) | |
| Phase B | | |
| Kronos 1001 | (4) CI 77891 (TITANIUM | 4.92 |
| | DIOXIDE) | |
| Unipure Yellow | (5) CI 77492 (IRON OXIDES) | 1.60 |
| LC 182 | | |
| Unipure Red LC | (5) CI 77491 (IRON OXIDES) | 0.20 |
| 381 | | |
| Unipure Brown | (5) CI 77491 (IRON OXIDES) | 0.20 |
| LC 889 | CI 77499 (IRON OXIDES) | |
| Unipure Blue LC | (5) CI 77007 (ULTRAMARINE | 0.08 |
| 686 | BLUE) | |
| Phase C | | |
| Miglyol 812N | (6) CAPRYLIC/CAPRIC | 7.00 |
| | TRIGLYCERIDE | |
| Eutanol G | (7) OCTYLDODECANOL | 4.00 |
| Montanov 202 | (8) ARACHIDYL ALCOHOL, | 4.00 |
| | BEHENYL ALCOHOL, | |
| | ARACHIDYLGLUCOSIDE | |
| Avocado oil | (9) PERSEA GRATISSIMA | 2.00 |
| | (AVOCADO OIL) | |
| Eusolex ® 9020 | (1) BUTYL | 1.50 |
| | METHOXYDIBENZOYL- | |
| | METHANE | |
| Hydrolite-5 | (10) PENTYLENE GLYCOL | 1.20 |
| Bentone gel | (11) STEARALKONIUM | 1.00 |
| GTCC V | HECTORITE, PROPYLENE | |
| | CARBONATE, | |
| | CAPRYLIC/CAPRIC | |
| | TRIGLYCERIDE | |
| RonaCare ® | (2) TOCOPHERYL ACETATE | 0.50 |
| all-rac-alpha- | | |
| tocopheryl | | |
| acetate | | |
| Phenonip | (12) PHENOXYETHANOL, | 0.40 |
| | BUTYLPARABEN, | |
| | ETHYLPARABEN, | |
| | PROPYLPARABEN, | |
| | METHYLPARABEN | |
| Oxynex ® K | (1) PEG-8, TOCOPHEROL, | 0.03 |
| liquid | ASCORBYL PALMITATE, | |
| | ASCORBIC ACID, CITRIC | |
| | ACID | |
| Phase D | | |
| Simulgel EG | (8) SODIUM ACRYLATE/ | 0.60 |
| | SODIUM ACRYLOYL- | |
| | DIMETHYLTAURATE | |
| | COPOLYMER, | |
| | ISOHEXADECANE, | |
| | POLYSORBATE 80 | |
| Phase E | | |
| Water, | AQUA (WATER) | 1.00 |
| demineralized | | |

### Preparation:

Add the Keltrol slowly to the water of phase A and disperse. Scatter in the remaining constituents of phase A with stirring. Add the constituents of phase B to phase A and homogenize using the Ultra-Turrax T25 (red-blue setting, 13500-20500 rpm) for 3 min and check for agglomerates. Heat phase A/B and phase C separately to 75° C. Add phase C to phase A/B with stirring and homogenize for 2 min using the Ultra-Turrax T25 (yellow-green setting, 8000-9500 rpm). Add phase D at between 55 and 60° C., add phase E at 40° C., and cool to room temperature with further stirring; adjust the pH to 7.0 using 30% citric acid. Then transfer into suitable containers. A light, slightly opaque foundation is obtained which is suitable for all skin types. Avocado oil, vitamin E acetate and cell-protecting RonaCare® ectoin support the skin-care action.

### Sources of Supply:

(1) Merck KGaA/Rona®
(2) C.P. Kelco
(3) BASF AG
(4) Kronos International Inc.
(5) Les Colorants Wackherr
(6) Sasol Germany GmbH
(7) Cognis GmbH
(8) Seppic
(9) Gustav Heess GmbH
(10) Symrise
(11) Elementis Specialities
(12) Clariant GmbH

### Example A5

### Body Lotion

| Raw material | INCI | [%] |
|---|---|---|
| Phase A | | |
| Aloe vera gel 10x | (1) ALOE BARBADENSIS | 2.00 |
| decolorized | | |
| D-Panthenol | (2) PANTHENOL | 0.40 |
| Pigment mixture | | 6.00 |
| from Example 3 | | |
| RonaCare ® | (3) ALLANTOIN | 0.20 |
| allantoin | | |
| Glycerin, | (3) GLYCERIN | 4.00 |
| anhydrous | | |
| Water, | AQUA (WATER) | 67.57 |
| demineralized | | |
| Phase B | | |
| Protelan AGL | (4) SODIUM COCOYL | 6.00 |
| 95/C | GLUTAMATE | |
| Cosmacol EMI | (5) DI-C12-13 ALKYL MALATE | 3.00 |
| Eutanol G | (6) OCTYLDODECANOL | 3.00 |
| Jojoba oil | (7) SIMMONDSIA CHINENSIS | 1.50 |
| | (JOJOBA OIL) | |
| Tegosoft TN | (8) C12-15 ALKYL BENZOATE | 1.50 |
| Carbopol ETD | (9) ACRYLATES/C10-30 ALKYL | 0.60 |
| 2020 | ACRYLATE CROSSPOLYMER | |
| Phenonip | (10) PHENOXYETHANOL, | 0.60 |
| | BUTYLPARABEN, | |
| | ETHYLPARABEN, | |
| | PROPYLPARABEN, | |
| | METHYLPARABEN | |
| RonaCare ® | (3) BISABOLOL | 0.50 |
| bisabolol | | |
| RonaCare ® | (3) TOCOPHERYLACETATE | 0.50 |
| all-rac-alpha- | | |
| tocopheryl | | |
| acetate | | |
| Oxynex ® ST | (3) DIETHYLHEXYL | 0.50 |
| liquid | SYRINGYLIDENEMALONATE, | |
| | CAPRYLIC/CAPRIC | |
| | TRIGLYCERIDE | |
| Cremophor RH | (11) PEG-40 HYDROGENATED | 0.30 |
| 410 | CASTOR OIL | |
| Oxynex ® K | PEG-8, TOCOPHEROL, | 0.03 |
| liquid | ASCORBYL PALMITATE, | |
| | ASCORBIC ACID, CITRIC ACID | |
| Phase C | | |
| Lifetime | (12) PERFUME | 0.50 |
| DH10255/1 | | |
| perfume oil | | |
| Phase D | | |
| Water, | AQUA (WATER) | 1.00 |
| demineralized | | |
| Germal 115 | (13) IMIDAZOLIDINYL UREA | 0.30 |

### Preparation:

Pre-dissolve the aloe vera and RonaCare® allantoin in the water of phase A with stirring, then add the other constituents of phase A and heat to 60° C. Introduce the jojoba oil, Oxynex K liquid, Cosmacol EMI, Eutanol G and Tegosoft TN into a stirred vessel, then incorporate the Carbopol homogeneously using the disperser disc (about 700 rpm, 20 min). Then add the remaining constituents of phase B, and stir everything to give a homogeneous mixture, only adding the Protelan AGL 95/C right at the end of phase B in order to prevent excessive incorporation of air. Slowly emulsify phase A into phase B (RT) at 60° C. with the aid of the disperser disc. Add phases C and D, then homogenize for 4 min using the Ultra-Turrax T50, speed 4. Cool to room temperature.

pH (23° C.) =5.5−6.0

Viscosity: Brookfield DV II+Helipath, spindle C, 5 rpm, 24° C.=11200 mPa·s

### Sources of Supply:

(1) Terry Laboratoires
(2) Alfa Aesar GmbH & Co. KG
(3) Merck KGaA/Rona®
(4) Zschimmer & Schwarz GmbH & Co.
(5) Nordmann, Rassmann GmbH & Co.
(6) Cognis GmbH
(7) Gustav Heess GmbH
(8) Evonik Goldschmidt GmbH
(9) Noveon
(10) Clariant GmbH
(11) BASF AG
(12) Parfex
(13) ISP Global Technologies

### Example A6

### Nail Varnish

| Raw material | INCI | [%] |
|---|---|---|
| Pigment mixture | (1) | 2.00 |
| from Example 8 | | |
| Thixotropic nail | (2) ETHYL ACETATE, BUTYL | 98.00 |
| varnish base 12897 | ACETATE, | |
| | NITROCELLULOSE, | |
| | PHTHALIC | |
| | ANHYDRIDE/TRIMETLLITIC | |
| | ANHYDRIDE/GLYCOLS | |
| | COPOLYMER, ACETYL | |
| | TRIBUTYL CITRATE, | |
| | ISOPROPYL ALCOHOL, | |
| | STEARALKONIUM | |
| | HECTORITE, ADIPIC | |
| | ACID/NEOPENTYL | |
| | GLYCOL/TRIMELLITIC | |
| | ANYHDRIDE COPLYMER | |

### Preparation:

The pigment mixture from Example 1 is weighed out together with the varnish base, mixed well and subsequently stirred at 1000 rpm for 10 minutes.

### Sources of Supply:

(1) Merck KGaA
(2) International Lacquers S.A.

### Example A7

### Lipstick

| Raw material | INCI | [%] |
|---|---|---|
| Phase A | | |
| Pigment mixture | (1) | 15.00 |
| from Example 4 | | |
| Phase B | | |
| Oxynex ® K | (1) PEG-8, TOCOPHEROL, | 0.05 |
| liquid | ASCORBYL PALMITATE, | |
| | ASCORBIC ACID, CITRIC | |
| | ACID | |
| Sensiva ® PA 20 | (2) PHENETHYL ALCOHOL, | 1.00 |
| | ETHYLHEXYL GLYCERIN | |
| Viscous paraffin | (1) PARAFFINUM LIQUIDUM | 2.10 |
| Adeps lanae | (3) LANOLIN | 3.50 |
| Paracera C 44 | (4) COPERNICIA CERIFERA, | 5.25 |
| | CERESIN | |
| Isopropyl myristate | (5) ISOPROPYL MYRISTATE | 5.60 |
| White wax | (1) CERA ALBA | 8.75 |
| Castor oil | (3) RICINUS COMMUNIS | 58.75 |
| | (CASTOR) SEED OIL | |

### Preparation:

Heat the constituents of phase B to about 75° C. until everything has melted Add phase A and stir well. Cool the lipstick composition to 65° C. and stir until the phase is free from air bubbles. Pour the homogeneous melt into the casting molds preheated to 55° C. Subsequently cool the molds and remove the cold castings. Warm the lipsticks to room temperature and then briefly flame-treat the lipsticks.

### Sources of Supply:

(1) Merck KGaA
(2) Schülke & Mayr GmbH
(3) Henry Lamotte Oils GmbHSasol Germany GmbH
(4) Azelis Germany GmbH
(5) BASF AG

The entire disclosures of all applications, patents and publications, cited herein and of corresponding European Application No. DE 102016004164.1, filed Apr. 11, 2016 are incorporated by reference herein.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A pigment mixture comprising at least two components A and B, where
component A is a mixture of flake-form and spherical substrates which is covered with one or more inorganic layers and/or organic layers,
and
component B comprises crystalline or amorphous particles selected from the group of the metal oxides, metal hydroxides, metal oxyhalides, Prussian Blue or mixtures thereof.

2. The pigment mixture according to claim 1, wherein said flake-form substrates are natural mica, synthetic mica, talc, kaolin, glass flakes, SiO₂ flakes, Al₂O₃ flakes, graphite flakes, Fe₂O₃ flakes, BiOCl, TiO₂ flakes, nitride flakes, oxynitride flakes, BN flakes, pearl essence or mixtures thereof.

3. The pigment mixture according to claim 1, wherein said spherical substrates are magnesium silicate, aluminum silicate, alkali metal aluminum silicate, alkaline-earth metal aluminum silicate and combinations thereof, SiO₂ spheres, glass spheres, hollow glass spheres, aluminum oxide, ceramic spheres and polymeric spheres comprising ethylene-acrylic acid copolymers, ethylene-methacrylate copolymers, HDI-trimethylolhexyl lactone copolymers, nylon, polyacrylates, polymethyl methacrylate copolymers, polyethylene, polymethylsilsesquioxanes or combinations thereof.

4. The pigment mixture according to claim 1, wherein component B consists of crystalline or amorphous particles of Fe₂O₃, TiO₂, Prussian Blue, Cr₂O₃, Fe₃O₄, FeOOH, BiOCl, titanium suboxides or mixtures thereof.

5. The pigment mixture according to claim 1, wherein said flake-form substrates of component A are natural or synthetic mica flakes.

6. The pigment mixture according to claim 1, wherein said spherical substrate of component A comprises SiO₂ spheres, glass spheres or hollow glass spheres.

7. The pigment mixture according to claim 1, wherein the substrate mixture of component A is covered with one or two metal oxide layers or mixtures of metal oxides.

8. The pigment mixture according to claim 1, wherein the inorganic layer of component A is TiO₂, Fe₂O₃, Fe₃O₄, FeOOH, Cr₂O₃, Prussian Blue, Carmine Red, titanium suboxide, SnO₂, ZnO, Al₂O₃, SiO₂ or mixtures thereof.

9. The pigment mixture according to claim 1, wherein component A has one of the following compositions:
substrate mixture+Fe₂O₃ (1st layer)+SnO₂ (2nd layer)
substrate mixture+Fe₂O₃ (1st layer)+SiO₂ (2nd layer)
substrate mixture+Fe₃O₄ (1st layer)+TiO₂ (2nd layer)
substrate mixture+Fe₃O₄ (1st layer)+SnO₂ (2nd layer)
substrate mixture+Fe₃O₄ (1st layer)+SiO₂ (2nd layer)
substrate mixture+Cr₂O₃ (1st layer)+TiO₂ (2nd layer)
substrate mixture+Cr₂O₃ (1st layer)+SnO₂ (2nd layer)
substrate mixture+Cr₂O₃ (1st layer)+SiO₂ (2nd layer)
substrate mixture+TiO₂ (1st layer)+Prussian Blue (2nd layer)
substrate mixture+TiO₂ (1st layer)+Carmine Red (2nd layer)
substrate mixture+SnO₂ (1st layer)+TiO₂ (2nd layer)
substrate mixture+TiO₂ (1st layer)
substrate mixture+Fe₂O₃ (1st layer)
substrate mixture+FeOOH (1st layer)
substrate mixture+TiO₂ (1st layer)+Fe₂O₃ (2nd layer)
substrate mixture+Prussian Blue (1st layer)
substrate mixture+Cr₂O₃ (1st layer)
substrate mixture+Fe₂O₃ (1st layer)+TiO₂ (2nd layer)+SiO₂ (3rd layer) or
substrate mixture+TiO₂ (1st layer)+SiO₂ (2nd layer)+TiO₂ (3rd layer).

10. The pigment mixture according to claim 1, wherein components A and B are mixed in the weight ratio (parts by weight) of 99:1 to 1:99.

11. The pigment mixture according to claim 1, wherein component A and/or component B additionally has an outer protective layer in order to increase the light, temperature and weather stability.

12. A process for the preparation of the pigment mixture according to claim 1, comprising: covering the flake-form and spherical substrates of component A with one or more inorganic layers by wet-chemical coating or by the CVD or PVD process, and subsequently mixing component A and component B with one another and subsequently drying and optionally jointly calcining.

13. The process according to claim 12, wherein the flake-form and spherical substrates of component A are mixed and covered with one or more absorbent or non-absorbent layers by wet-chemical methods, and the coated substrate/sphere mixture (component A) is mixed with the crystalline or amorphous particles (component B), and the mixture of components A and B is worked up jointly, and the final mixture is calcined at temperatures of 150-1000° C., where the calcination is optionally carried out under reducing gas.

14. The process according to claim 12, wherein, in the case of wet-chemical coating, the aqueous suspension of component B is added to the aqueous suspension of component A and the suspension comprising components A and B is worked up, or in that, after the wet-chemical covering of the substrate mixture of component A, the product is separated off and worked up and the dried product of component A is subsequently mixed with the suspension of component B and the suspension is then worked up, or in that the substrate mixture of component A is added to the suspension of component B and the mixture of component B and substrate mixture of component A is jointly covered with one or more inorganic layers by wet-chemical methods and then worked up.

15. Use of the pigment mixture according to claim 1 in paints, coatings, printing inks, security printing inks, plastics, ceramic materials, glazes, glasses, in cosmetic formulations, as tracer, as filler and for the preparation of pigment preparations and dry preparations.

16. A formulation comprising a pigment mixture according to claim 1.

17. The formulation according to claim 16, wherein, besides the pigment mixture, said formulation comprises at least one constituent selected from absorbents, astringents, antimicrobial substances, antioxidants, antiperspirants, antifoaming agents, antidandruff active compounds, antistatics, binders, biological additives, bleaches, chelating agents, deodorizers, emollients, emulsifiers, emulsion stabilizers, dyes, humectants, film formers, fillers, fragrances, flavors, insect repellents, preservatives, anticorrosion agents, cosmetic oils, solvents, oxidants, vegetable constituents, buffer substances, reducing agents, surfactants, propellant gases, opacifiers, UV filters and UV absorbers, denaturing agents, aloe vera, avocado oil, coenzyme Q10, green tea extract, viscosity regulators, perfume and vitamins.
